(19) United States
(12) Patent Application Publication
(10) Pub. No.: US 2024/0345070 A1
(43) Pub. Date: Oct. 17, 2024

(54) METHODS FOR PREDICTING AND/OR MONITORING CANCER TREATMENT RESPONSE USING CHANGES IN CIRCULATING CANCER ASSOCIATED MACROPHAGE-LIKE CELLS (CAMLS)

(71) Applicant: Creatv MicroTech, Inc., Potomac, MD (US)

(72) Inventors: Daniel L. ADAMS, Basking Ridge, NJ (US); Cha-Mei TANG, Potomac, MD (US)

(73) Assignee: Creatv MicroTech, Inc., Potomac, MD (US)

(21) Appl. No.: 18/580,301

(22) PCT Filed: Jul. 21, 2022
(86) PCT No.: PCT/US2022/037874

§ 371 Date: Jan. 18, 2024

Related U.S. Application Data
(60) Provisional application No. 63/317,642, filed on Mar. 8, 2022.
(60) Provisional application No. 63/240,669, filed on Sep. 3, 2021.
(60) Provisional application No. 63/224,166, filed on Jul. 21, 2021.

(51) Int. Cl. G01N 33/50; A61B 5/00; G01N 33/574; G16H 10/40

(52) U.S. Cl. CPC G01N 33/5091; A61B 5/4848; A61B 5/7275; G01N 33/57492; G16H 10/40; G01N 2333/70532

(57) ABSTRACT

Means for predicting treatment response in a subject having cancer are disclosed, where the predictions are based comparing the number and size of circulating cancer associated macrophage-like cells (CMLS) and circulating tumor cells (CTCs) found in biological samples at baseline and after induction of therapy, such as blood, from the subject.

### BACKGROUND

### Field of the Invention

The present invention generally relates to the use of biomarkers in the blood and other bodily fluids to make predictions and/or monitor cancer treatment response in subjects having cancer, such as solid tumors. The information can be useful to patients and oncologists in guiding cancer treatment.

### Related Art

When tumor cells break away from primary solid tumors, they penetrate into the blood or lymphatic circulation, and ultimately leave the blood stream and enter either organs or tissue to form metastasis. 90% of cancer-related deaths are caused by the metastatic process. The most common metastatic sites are the lung, liver, bone and brain. Tumor cells found in the circulation are called circulating tumor cells (CTCs). Many research publications and clinical trials show that CTCs have clinical utility in (i) providing prognostic survival and cancer recurrence information through the enumeration of CTCs in the blood stream, and (ii) providing treatment information through examination of protein expression levels, and the occurrence of gene mutations and translocations in the CTCs. However, CTCs are not consistently associated with the development and/or presence of cancer in a subject, even in stage IV cancer patients. While CTCs are found most often in stage IV of breast, prostate and colorectal cancers, they are rare in early stages of the same cancer. CTCs are also rare in other cancers.

Circulating cancer associated macrophage-like cells (CAMLs) are another cancer-related cell type that is found in the blood of subjects having cancer. CAMLs are associated with all solid tumors tested and all stages of cancer. CAMLs are polyploid and very large in size, ˜25 μm to ˜300 μm in size, or in some cases, even larger. These polyploid cells can be CD45 (−) or (+), and they typically express CD14 and CD31, which confirms their origin as a myeloid lineage. They are often found in the process of engulfing CTCs and cell debris [1-6].

Assays associated with the identification and characterization of biomarkers, such as CAMLs, in blood and other body fluids can be used to provide predictive and prognostic information. The present invention is directed to providing such tools to clinicians and other important goals.

### SUMMARY

The present invention is directed methods of using a type of cell with unique characteristics that is found in the blood of subjects having solid tumors, including carcinoma, sarcoma, neuroblastoma and melanoma. These circulating cells, termed “circulating Cancer Associated Macrophage-like cells” (CAMLs), have been shown to be associated with the presence of solid tumors in a subject having cancer. Five morphologies associated with CAMLs have been characterized and described [1-3]. CAMLs have been found consistently in the peripheral blood of subjects having stage I to stage IV solid tumors by size exclusion microfiltration using precision microfilters.

Medical applications associated with CAMLs include, but are not limited to, use of the cells as a biomarker to provide early detection of cancer and diagnosis of cancer, in particular, in the early detection and diagnosis of cancer relapse or recurrence, and in the determination of cancer mutation. The size of CAMLs has been shown to provide prognostic information. Patients with CAMLs larger than 50 μm, in samples of 7.5 mL of blood, were shown to have short progression free survival and overall survival compared to patients with no CAMLs larger than 50 μm [4].

As reported herein for the first time, other properties associated with CAMLs that are found in the blood of cancer patients, as well as properties associated with circulating tumor cells (CTCs) found in the same samples, can be used to provide both predictive and prognostic information critical to patient care. For example, when clinical data obtained from a given treatment regimen includes both a biomarker positive group and a biomarker negative group, then the data: (1) can be predictive, if the biomarker negative group cannot stratify the treatment responders versus non-responders, and (2) can be both predictive and prognostic, if the biomarker can stratify the responders in both biomarker positive and biomarker negative cohorts [7].

The present invention takes advantage of these properties and provides, in a first embodiment, methods for predicting a treatment response in a subject having cancer by determining and comparing the size and number of CAMLs in at least two samples from the same patient, wherein a first patient sample is obtained before therapy (i.e. a pre-treatment sample) and a second patient sample is obtained after therapy (i.e. a post-treatment sample). A baseline (BL) is provided by the first (pre-treatment) patient sample. In certain aspects of the invention, when at least one CAML in the post-treatment sample is greater in size than the largest CAML in the pre-treatment sample and wherein there are more CAMLs in the post-treatment sample than in the pre-treatment sample, the subject is predicted to not respond to treatment.

A first specific aspect of this embodiment is a method for predicting a treatment response in a subject having cancer comprising obtaining at least one pre-treatment sample from a subject having cancer and obtaining at least one post-treatment sample from the subject, and comparing the number and size of CAMLs between the pre-and post-treatment samples for differences, wherein when a difference in CAML number and/or size is found, a treatment response is predicted. The difference may be a change in the size of the CAMLs, a change in the number of CAMLs, or both. The change in the size of the CAMLs may be a change in the average size calculated for all CAMLs in the samples, or the change in the size of the largest CAML in the samples. The change in the size may be an increase in size or a decrease in size.

A second specific aspect of this embodiment is a method for predicting a treatment response in a subject having cancer comprising obtaining at least one pre-treatment sample from a subject having cancer and obtaining at least one post-treatment sample from the subject, and comparing the number and size of CAMLs between the pre-and post-treatment samples for differences, wherein when the largest CAML in the post-treatment sample is decreased in size or similar in size in comparison to the largest CAML in the pre-treatment sample, and the number of CAMLs in the post-treatment sample is less than the number of CAMLs in the pre-treatment sample, the subject has a probability of benefiting from the treatment, or responding to the treatment.

A third specific aspect of this embodiment is a method for predicting a treatment response in a subject having cancer comprising obtaining at least one pre-treatment sample from a subject having cancer and obtaining at least one post-treatment sample from the subject, and comparing the number and size of CAMLs between the pre-and post-treatment samples for differences, wherein when the largest CAML in the post-treatment sample is decreased in size or similar in size in comparison to the largest CAML in the pre-treatment sample, and the number of CAMLs in the post-treatment sample is more than or the same as the number of CAMLs in the pre-treatment sample, the subject has a probability of not benefiting from the treatment, or not responding to the treatment.

A fourth specific aspect of this embodiment is a method for predicting a treatment response in a subject having cancer comprising obtaining at least one pre-treatment sample from a subject having cancer and obtaining at least one post-treatment sample from the subject, and comparing the number and size of CAMLs between the pre-and post-treatment samples for differences, wherein when the largest CAML in the post-treatment sample is increased in size or similar in size in comparison to the largest CAML in the pre-treatment sample, and the number of CAMLs in the post-treatment sample is less than or the same as the number of CAMLs in the pre-treatment sample, the subject has a probability of not benefiting from the treatment, or not responding to the treatment.

A fifth specific aspect of this embodiment is a method for predicting a treatment response in a subject having cancer comprising obtaining at least one pre-treatment sample from a subject having cancer and obtaining at least one post-treatment sample from the subject, and comparing the number and size of CAMLs between the pre-and post-treatment samples for differences, wherein when the largest CAML in the post-treatment sample is increased in size or similar in size in comparison to the largest CAML in the pre-treatment sample, and the number of CAMLs in the post-treatment sample is more than the number of CAMLs in the pre-treatment sample, the subject has a probability of not benefiting from the treatment, or not responding to the treatment.

In a second embodiment, the invention is directed to methods for predicting a treatment response in a subject having cancer by assaying for CTCs in samples from a subject having cancer, wherein the presence of one or more CTCs in the sample following cancer treatment (i.e. post-treatment sample) suggests the subject has a probability of not benefiting from the treatment, or responding to the treatment. While CTCs are rarely found in the blood of cancer patients, except in metastatic breast, prostate and colorectal cancers and small cell lung cancer (SCLC), confirming their presence in some samples can be predictive of treatment response.

One specific aspect of this embodiment is a method for predicting a treatment response in a subject having cancer comprising obtaining at least one post-treatment sample from a subject having cancer, and assaying the sample for CTCs, wherein when one or more CTCs are detected in the sample, the subject has a probability of not benefiting from the treatment, or not responding to the treatment.

In a third embodiment, the invention is directed to methods that combine the features of the first and second embodiments.

The methods of this third embodiment can also be used for predicting a treatment response in a subject having cancer. The method comprises (i) comparing the size and number of CAMLs in at least two patient samples, wherein a first patient sample is obtained before therapy (i.e. a pre-treatment sample) and a second patient sample is obtained after therapy (i.e. a post-treatment sample), and (ii) determining the number of CTCs in the second patient sample obtained after therapy (i.e. a post-treatment sample).

One specific aspect of this embodiment is a method for predicting a treatment response in a subject having cancer comprising obtaining at least one pre-treatment sample from a subject having cancer and obtaining at least one post-treatment sample from the subject, and (i) comparing the number and size of CAMLs between the pre-and post-treatment samples for differences and (ii) determining the number of CTCs in the post-treatment samples, wherein when a difference in CAML number and/or size is found and/or at least one CTC is detected, a treatment response is predicted. As an example, when at least one CTC is detected in the post-treatment sample, the subject has a probability of not benefiting from the treatment, or not responding to the treatment.

As in the embodiments above, the difference may be a change in the size of the CAMLs, a change in the number of CAMLs, or both. The change in the size of the CAMLs may be a change in the average size calculated for all CAMLs in the samples, or the change in the size of the CAMLs may be a change in the size of the largest CAML in the samples. The change in the size may be an increase in size or a decrease in size.

In each of the embodiments and aspects of the invention, the CAMLs can be defined as having each of the following characteristics:
(a) large atypical polyploid nucleus of about 14-64 μm in size, or larger, or multiple nuclei in a single cell;
(b) cell size of about 20-300 μm in size, though larger is possible; and
(c) morphological shape selected from the group consisting of spindle, tadpole, round, oblong, two legs, thin legs, and amorphous.

In certain aspects of the embodiments of the invention, the CAMLs can be further defined as possibly having one or more of the following additional characteristics:
(d) CD14 positive phenotype;
(e) CD45 expression;
(f) EpCAM expression;
(g) vimentin expression;
h) PD-L1 expression;
(i) monocytic CD11C marker expression;
(j) endothelial CD146 marker expression;
(k) endothelial CD202b marker expression; and
(l) endothelial CD31 marker expression.

In each aspect and embodiment of the invention, the sample is a biological sample and the sample may be, but is not limited to, one or more of peripheral blood, blood, lymph node, bone marrow, cerebral spinal fluid, tissue, urine, peripheral blood mononuclear cells (PBMCs), and cryopreserved PBMCs. When the biological sample is blood, the blood may be antecubital-vein blood, inferior-vena-cava blood, femoral vein blood, portal vein blood, or jugular-vein blood, for example. The sample may be a fresh sample or a properly prepared cryo-preserved sample that is thawed. In certain aspects of the invention, the sample is a blood sample and the size of the blood sample is between 5 and 50 mL.

In each aspect and embodiment of the invention, the cancer is a solid tumor, Stage I cancer, Stage II cancer, Stage III cancer, Stage IV cancer, carcinoma, sarcoma, neuroblastoma, melanoma, epithelial cell cancer, breast cancer, prostate cancer, lung cancer, pancreatic cancer, colorectal cancer, liver cancer, head and neck cancer, kidney cancer, ovarian cancer, esophageal cancer, uterine cancer, urothelial cancer, bladder cancer, endometrial cancer, cholangiocarcinoma, neuroendocrine cancer or other solid tumor cancer.

In certain aspects of the embodiments of the invention, the CAMLs and CTCs may be isolated from the samples using one or more means selected from size exclusion methodology, immunocapture, dendrimer-mediated multivalent cell capture, affinity based surface capture, biomimetic surface coating capture, selectin coated surfaces capture, other functionalized surface captures, inertial focusing chips, red blood cell lysis, white blood cell depletion, FICOLL separation, electrophoresis, dielectrophoresis, flow cytometry, magnetic levitation, and various microfluidic chips, or a combination thereof.

In one aspect of the invention, the CAMLs and CTCs may be isolated from the samples using size exclusion methodology that comprises using a microfilter. The microfilter may have a pore size ranging from about 5 microns to about 10 microns to capture both CTCs and CAMLs, and the microfilter may have a pore size ranging to about 20 microns to capture mainly CAMLs. The pores of the microfilter may have a round, race-track shape, oval, square and/or rectangular pore shape. The microfilter may have precision pore geometry and/or uniform pore distribution.

In another aspect of the invention, the CAMLs and CTCs may be isolated from the samples using a microfluidic chip via physical size-based sorting, hydrodynamic size-based sorting, grouping, trapping, immunocapture, concentrating large cells, or eliminating small cells based on size.

In a further aspect of the invention, the CAMLs and CTCs may be isolated from the biological samples using a CellSieveTM low-pressure microfiltration assay.

### BRIEF DESCRIPTION OF THE DRAWINGS

FIG. 1 illustrates changes of CAML size and number at post-treatment from the pre-treatment sample. (i) When the CAMLs in the post-treatment sample have an increase of CAML number and the size of the largest CAML has increased from the pre-treatment sample, the patient is designated as “increase in CAMLs”. (ii) When the CAMLs in the post-treatment sample have an increase of CAML number and the size of the largest CAML does not show an increase from the pre-treatment sample, the patient is designated as “mixed in CAMLs”. (iii) When the CAMLs in the post-treatment sample do not have an increase in number and the size of the largest CAML shows an increase from the cells of the pre-treatment sample, the patient is also designated as “mixed in CAMLs”. (iv) When the CAMLs in the post-treatment sample have a decrease in number and the size of the largest CAML has decreased from the pre-treatment sample, the patient is designated as “reduction in CAMLs”.

FIG. 2A shows the stratification of progression free survival (PFS) stratified based on the change of CAML size or number for one drug treatment study.

FIG. 2B shows the stratification of overall survival (OS) stratified based on the change of CAML size or number for one drug treatment study.

FIG. 3 shows spider plots of change of tumor size for a group of metastatic breast cancer patients following treatment based on change in CAML size or number.

FIG. 4 shows the Kaplan-Meier plot of PFS of a larger cohort of metastatic breast cancer patients, some of which were shown in the spider plot in FIG. 3, taking in the account the changes of CAML size and/or number.

FIG. 5A shows the Kaplan-Meier plot of PFS of patients with metastatic breast cancers taking into account CAML size and number and presence of CTCs, comparing CTC and CAML information at baseline and after therapy.

FIG. 5B shows the Kaplan-Meier plot of OS of patients with metastatic breast cancers taking into account CAML size and number and presence of CTCs, comparing CTC and CAML information at baseline and after therapy.

FIG. 6A shows the Kaplan-Meier plot of PFS of 220 metastatic cancer patients with a number of different cancer types, taking into account CAML size and number and presence of CTCs, comparing CTC and CAML information at baseline and after therapy.

FIG. 6B shows the Kaplan-Meier plot of OS of 220 metastatic cancer patients with a number of different cancer types, taking into account CAML size and number and presence of CTCs, comparing CTC and CAML information at baseline and after therapy.

FIG. 7A shows the Kaplan-Meier plot of PFS of 220 metastatic cancer patients with a number of different cancer types taking in the account patients with >50 μm CAMLs and presence of CTCs after therapy.

FIG. 7B shows the Kaplan-Meier plot of OS of 220 metastatic cancer patients with a number of different cancer types taking in the account patients with >50 μm CAMLs and presence of CTCs after therapy.

FIG. 8 shows stratification of (A) PFS and (B) OS of 25 triple negative breast cancer (TNBC) patients analyzed 30 days after treatment with CCR5 drug, based on changes of CAML size and number from the baseline

FIG. 9 shows stratification of (A) PFS and (B) OS of 25 triple negative breast cancer (TNBC) patients analyzed 30 days after treatment with CCR5 drug. Four additional TNBC patients treated with CCR5 drug, but did not have data at 30 days after treatment are include. For these four patients, patients with CAMLs <50 μm at baseline are group with the CAML decreasing group.

FIG. 10 shows PFS and OS of changes in CAML size by 35 μm. (a) & (b) PFS & OS for all n=182 NSCLC patients. (c) & (d) PFS & OS for IMT population (n=91, green) and CRT alone population (n=91)

FIG. 11 shows effect of CXCR4 expression in CTCs, CAMLs and epithelial mesenchymal transition cells (EMTs). Kaplan-Meier graphs of PFS and OS for CTCs, CAMLs, and EMT expression. (a.) PFS of CXCR4 expression on CTCs. (b.) OS of CXCR4 expression on CTCs (c.) PFS of CXCR4 expression on CAMLs. (d.) OS of CXCR4 expression on CAMLs. (e.) PFS of CXCR4 expression on EMTs. (f.) OS of CXCR4 expression on EMTs.

FIG. 12 shows stratification of (A) PFS and (B) OS of NSCLC patients treated with chemoradiation therapy (CRT) followed by immunotherapy comparing PD-L1 expression in CAMLs.

FIG. 13 shows stratification of (A) PFS and (B) OS of NSCLC patients treated with chemoradiation therapy (CRT) followed by immunotherapy by grouping PD-L1 expression combined with CAML size. The non-responders belong to the group of patients with low PD-L1 and one or more CAMLs ≥50 μm.

FIG. 14 shows stratification of (A) PFS and (B) OS of NSCLC patients receiving chemoradiation therapy (CRT) with (n=41) or without (n=41) additional anti-PD-L1/PD-1 immunotherapy comparing high and low PD-L1 expression in CAMLs.

FIG. 15 shows stratification of (A) PFS and (B) OS of NSCLC patients receiving chemoradiation therapy (CRT) with (n=41) or without (n=41) additional anti-PD-L1/PD-1 immunotherapy comparing changes of PD-L1 expression in CAMLs between pre-treatment and post-treatment.

### DETAILED DESCRIPTION

The matters defined in the description such as a detailed construction and elements are nothing but the ones provided to assist in a comprehensive understanding of the invention. Accordingly, those of ordinary skill in the art will recognize that various changes and modifications of the embodiments described herein can be made without departing from the scope and spirit of the invention.

Cancer is one of the most feared illness in the world, affecting all populations and ethnicities in all countries. Approximately 40% of both men and women will develop cancer in their lifetime. In the United States alone, at any given time there are more than 12 million cancer patients, with 1.7 million new cancer cases and more than 0.6 million deaths estimated for 2018. Cancer death worldwide is estimated to be about 8 million annually, of which 3 million occur in developed countries where patients have access to treatment.

Ideally, there will be diagnostics that can quickly determine if a selected therapy is working. Ideally, the diagnostics is a blood test.

In this disclosure, a cell type is presented that is more consistently found in the blood of solid tumor patients from Stages I-IV than any other cancer related cell. These circulating cells are macrophage-like cells that contain the same tumor markers as the primary tumor and they are termed circulating Cancer Associated Macrophage-like cells (CAMLs) herein.

Along with circulating tumor cells (CTCs), CAMLs present in biological samples from patients having cancer can be isolated and characterized, for example through the use of size exclusion methods, including microfiltration methods. Microfilters can be formed with pores large enough to allow red blood cells and the majority of white blood cells to pass, while retaining larger cells such as CTCs and CAMLs. The collected cells can then be characterized, either directly on the filters or through other means.

CAMLs have many clinical utilities when used alone. Furthermore, the characterization of CAMLs in a biological sample can be combined with the assaying of other markers such as CTCs, cell-free DNA and free proteins in blood to further improve sensitivity and specificity of a diagnosis technique. This is especially true for CAMLs and CTCs because they can be isolated and identified at the same time using the same means.

### Circulating Cancer Associated Macrophage-Like Cells (CAMLs)

As defined herein, the circulating cells used in the methods of the invention can be termed “CAMLs”. CAMLs are characterized by having one or more of the following features:
CAMLs have a large, atypical polyploid nucleus or multiple individual nuclei, often scattered in the cell, though enlarged fused nucleoli are common. CAML nuclei generally range in size from about 10 μm to about 70 μm in diameter, more commonly from about 14 μm to about 64 μm in diameter.
CAMLs can be CD45 positive or CD45 negative, and the present invention encompasses the use of both types of CAMLs.
CAMLs are large, approximately 20 micron to approximately 300 micron in size by the longest dimension.
CAMLs are found in many distinct morphological shapes, including spindle, tadpole, round, oblong, two legs, more than two legs, thin legs, or amorphous shapes.
CAMLs from carcinomas typically have diffused cytokeratins.
If CAMLs express EpCAM, EpCAM is typically diffused throughout the cell, or associated with vacuoles and/or ingested material, and nearly uniform throughout the whole cell, but not all CAML express EpCAM, because some tumors express very low or no EpCAM.
If CAMLs express a marker, the marker is typically diffused throughout the cell, or associated with vacuoles and/or ingested material, and nearly uniform throughout the whole cell, but not all CAML express the same markers with equal intensity.
CAMLs often express markers associated with the markers of the tumor origin. For example, if the tumor is of prostate cancer origin and expresses PSMA, then CAMLs from such a patient also expresses PSMA. As another example, if the primary tumor is of pancreatic origin and expresses PDX-1, then CAMLs from such a patient also expresses PDX-1. As further example, if the primary tumor or CTC of the cancer origin express CXCR-4, then CAMLs from such a patient also express CXCR-4. CAMLs associated with epithelial cancers may express CK 8, 18 or 19, vimentin, etc. For sarcomas, neuroblastomas and melanomas, other markers associated with the cancers can be used instead of CK 8, 18, 19.
If the primary tumor or CTC originating from the cancer expresses a biomarker of a drug target, CAMLs from such a patient also express the biomarker of the drug target. An example of such a biomarker of immunotherapy is PD-L1.
CAMLs express monocytic markers (e.g. CD11c, CD14) and endothelial markers (e.g. CD146, CD202b, CD31).
CAMLs have the ability to bind Fc fragments.

CAMLs can be visualized by colorimetric stains, such as H&E, or fluorescent staining of specific markers. For the cytoplasm, CD31 is the most positive phenotype. CD31 alone, or in combination with other positive markers, or cancer markers associated with the tumor are recommended.

In the various embodiments and aspects of the invention, CAMLs can be defined as cells having the following characteristics: (a) a large atypical polyploid nucleus of about 14-64 um in size, or multiple nuclei in a single cell; (b) cell size of about 20-300 microns in size; and (c) a morphological shape selected from the group consisting of spindle, tadpole, round, oblong, two legs, more than two legs, thin legs, and amorphous. In further embodiments, the CAMLs can be defined as also having one or more of the following additional characteristics: (d) CD14 positive phenotype; (e) CD45 expression; (f) EpCAM expression; (g) vimentin expression; (h) PD-L1 expression; (i) monocytic CD11C marker expression; (j) endothelial CD146 marker expression; (k) endothelial CD202b marker expression; and (l) endothelial CD31 marker expression.

### Circulating Tumor Cells

As defined herein, CTCs associated with carcinomas express a number of cytokeratins (CKs). CK 8, 18, & 19 are the cytokeratins most commonly expressed and used in diagnostics, but surveying need not be limited to these markers alone. The surface of solid tumor CTCs usually express epithelial cell adhesion molecule (EpCAM). However, this expression is not uniform or consistent. CTCs do not express any CD45 because it is a white blood cell marker. In assays to identify tumor-associated cells, such as CTCs and CAMLs, it is sufficient to use antibodies against markers associated with the solid tumor such as CK 8, 18, & 19, or antibodies against CD45 or DAPI. Combining staining techniques with morphology, pathologically-definable CTCs (PDCTC), apoptotic CTCs and CAMLs can be identified [3].

PDCTCs associated with solid tumors express CK 8, 18, & 19, and can be identified and defined by the following characteristics:
A “cancer-like” nuclei stained by DAPI. The nuclei are usually large with dot patterns. The exception is when the cell is in division. The nucleus can also be condensed.
Expression of one or more of CK 8, 18 and 19; CTCs from epithelial cancers usually express at least CK 8, 18 and 19. The cytokeratins have a filamentous pattern.
Lack of CD45 expression.

An apoptotic CTCs associated with cancer express CK 8, 18, & 19 and can be identified and defined by the following characteristics:
A degrading nuclei.
Expression of one or more of CK 8, 18 and 19; not all the cytokeratins are filamentous in pattern, but parts or whole appear fragmented in the form of spots.
Lack of CD45 expression.

As suggested above, the unique characteristics of the CAMLs and CTCs described herein make them well-suited for use in clinical methodology including methods of screening and diagnosis diseases such as cancer, monitoring treatment, monitoring of disease progression and recurrence, and predicting treatment response.

### Methods for Predicting Treatment Response

The present invention takes advantage of these properties and provides, in a first embodiment, methods for predicting a treatment response in a subject having cancer by determining and comparing the size and number of CAMLs in at least two samples from the same patient, wherein a first patient sample is obtained before therapy (i.e. a pre-treatment sample) and a second patient sample is obtained after therapy (i.e. a post-treatment sample). A baseline (BL) is provided by the first (pre-treatment) patient sample. In certain aspects of the invention, when at least one CAML in the post-treatment sample is greater in size than the largest CAML in the pre-treatment sample and wherein there are more CAMLs in the post-treatment sample than in the pre-treatment sample, the subject is predicted to not respond to treatment.

A first specific aspect of this embodiment is a method for predicting a treatment response in a subject having cancer comprising obtaining at least one pre-treatment sample from a subject having cancer and obtaining at least one post-treatment sample from the subject, and comparing the number and size of CAMLs between the pre-and post-treatment samples for differences, wherein when a difference in CAML number and/or size is found, a treatment response is predicted. The difference may be a change in the size of the CAMLs, a change in the number of CAMLs, or both. The change in the size of the CAMLs may be a change in the average size calculated for all CAMLs in the samples, or the change in the size of the largest CAML in the samples. The change in the size may be an increase in size or a decrease in size.

A second specific aspect of this embodiment is a method for predicting treatment response in a subject having cancer comprising obtaining at least one pre-treatment sample from a subject having cancer and obtaining at least one post-treatment sample from the subject, and comparing the number and size of CAMLs between the pre-and post-treatment samples for differences, wherein when the largest CAML in the post-treatment sample is decreased in size or similar in size in comparison to the largest CAML in the pre-treatment sample, and the number of CAMLs in the post-treatment sample is less than the number of CAMLs in the pre-treatment sample, the subject has a probability of benefiting from the treatment, or responding to the treatment.

A third specific aspect of this embodiment is a method for predicting a treatment response in a subject having cancer comprising obtaining at least one pre-treatment sample from a subject having cancer and obtaining at least one post-treatment sample from the subject, and comparing the number and size of CAMLs between the pre-and post-treatment samples for differences, wherein when the largest CAML in the post-treatment sample is decreased in size or similar in size in comparison to the largest CAML in the pre-treatment sample, and the number of CAMLs in the post-treatment sample is more than or the same as the number of CAMLs in the pre-treatment sample, the subject has a probability of not benefiting from the treatment, or not responding to the treatment.

A fourth specific aspect of this embodiment is a method for predicting a treatment response in a subject having cancer comprising obtaining at least one pre-treatment sample from a subject having cancer and obtaining at least one post-treatment sample from the subject, and comparing the number and size of CAMLs between the pre-and post-treatment samples for differences, wherein when the largest CAML in the post-treatment sample is increased in size or similar in size in comparison to the largest CAML in the pre-treatment sample, and the number of CAMLs in the post-treatment sample is less than or the same as the number of CAMLs in the pre-treatment sample, the subject has a probability of not benefiting from the treatment, or not responding to the treatment.

A fifth specific aspect of this embodiment is a method for predicting a treatment response in a subject having cancer comprising obtaining at least one pre-treatment sample from a subject having cancer and obtaining at least one post-treatment sample from the subject, and comparing the number and size of CAMLs between the pre-and post-treatment samples for differences, wherein when the largest CAML in the post-treatment sample is increased in size or similar in size in comparison to the largest CAML in the pre-treatment sample, and the number of CAMLs in the post-treatment sample is more than the number of CAMLs in the pre-treatment sample, the subject has a probability of not benefiting from the treatment, or not responding to the treatment.

In a second embodiment, the invention is directed to methods for predicting a treatment response in a subject having cancer by assaying for CTCs in samples from a subject having cancer, wherein the presence of one or more CTCs in the sample following cancer treatment (i.e. post-treatment sample) suggests the subject has a probability of not benefiting from the treatment, or responding to the treatment. While CTCs are rarely found in the blood of cancer patients, except in metastatic breast, prostate and colorectal cancers and small cell lung cancer (SCLC), confirming their presence in some samples can be predictive of treatment response.

One specific aspect of this embodiment is a method for predicting a treatment response in a subject having cancer comprising obtaining at least one post-treatment sample from a subject having cancer, and assaying the sample for CTCs, wherein when one or more CTCs are detected in the sample, the subject has a probability of not benefiting from the treatment, or not responding to the treatment.

In a third embodiment, the invention is directed to methods that combine the features of the first and second embodiments.

The methods of this third embodiment can also be used for predicting a treatment response in a subject having cancer. The method comprises (i) comparing the size and number of CAMLs in at least two patient samples, wherein a first patient sample is obtained before therapy (i.e. a pre-treatment sample) and a second patient sample is obtained after therapy (i.e. a post-treatment sample), and (ii) determining the number of CTCs in the second patient sample obtained after therapy (i.e. a post-treatment sample).

One specific aspect of this embodiment is a method for predicting a treatment response in a subject having cancer comprising obtaining at least one pre-treatment sample from a subject having cancer and obtaining at least one post-treatment sample from the subject, and (i) comparing the number and size of CAMLs between the pre-and post-treatment samples for differences and (ii) determining the number of CTCs in the post-treatment samples, wherein when a difference in CAML number and/or size is found and/or at least one CTC is detected, a treatment response is predicted. In one aspect of this embodiment, when at least one CTC is detected in the post-treatment sample, the subject has a probability of not benefiting from the treatment, or not responding to the treatment.

As in the embodiments above, the difference may be a change in the size of the CAMLs, a change in the number of CAMLs, or both. The change in the size of the CAMLs may be a change in the average size calculated for all CAMLs in the samples, or the change in the size of the CAMLs may be a change in the size of the largest CAML in the samples. The change in the size may be an increase in size or a decrease in size.

In a further aspect of the invention, the level of PD-L1 expression can also be determined in the CAMLs and/or CTCs collected in the pre-and post-treatment samples. Experimental evidence provided herein and discussed below demonstrates that PD-L1 expression in CAMLs can also provide information useful in predicting treatment response in a subject having cancer, particularly in subjects being treated by immunotherapy. In general, the greater the level of PD-L1 expression in CAMLs, the higher the probability the subject will benefit from the treatment or respond to the treatment. Thus, the methods outlined in each of the embodiments defined herein can also be practiced by including a step of comparing the level of PD-L1 expression in CAMLs between the pre-and post-treatment samples for differences. In one aspect, when the level of PD-L1 expression in CAMLs in the post-treatment sample is lower than the expression level in the pre-treatment sample, the subject has a probability of not benefiting from the treatment, or not responding to the treatment. In another aspect, when the level of PD-L1 expression in CAMLs in the post-treatment sample is higher than the expression level in the pre-treatment sample, the subject has a probability of benefiting from the treatment, or responding to the treatment.

In a further aspect of the invention, the level of CXCR4 expression can also be determined in the CAMLs and/or CTCs collected in the pre-and post-treatment samples. Experimental evidence provided herein and discussed below demonstrates that CXCR4 expression in CAMLs can also provide information useful in predicting treatment response in a subject having cancer. In general, the lower the level of CXCR4 expression in CAMLs, the higher the probability the subject will benefit from the treatment or respond to the treatment. Thus, the methods outlined in each of the embodiments defined herein can also be practiced by including a step of comparing the level of CXCR4 expression in CAMLs between the pre-and post-treatment samples for differences. In one aspect, when the level of CXCR4 expression in CAMLs in the post-treatment sample is higher than the expression level in the pre-treatment sample, the subject has a probability of not benefiting from the treatment, or not responding to the treatment. In another aspect, when the level of CXCR4 expression in CAMLs in the post-treatment sample is lower than the expression level in the pre-treatment sample, the subject has a probability of benefiting from the treatment, or responding to the treatment.

In each of the embodiments and aspects of the invention, the CAMLs can be defined as having each of the following characteristics:
(a) large atypical polyploid nucleus of about 14-64 μm in size, or larger, or multiple nuclei in a single cell;
(b) cell size of about 20-300 μm in size, though larger is possible; and
(c) morphological shape selected from the group consisting of spindle, tadpole, round, oblong, two legs, thin legs, and amorphous.

In certain aspects of the embodiments of the invention, the CAMLs can be further defined as possibly having one or more of the following additional characteristics:
(d) CD14 positive phenotype;
(e) CD45 expression;
(f) EpCAM expression;
(g) vimentin expression;
(h) PD-L1 expression;
(i) monocytic CD11C marker expression;
(j) endothelial CD146 marker expression;
(k) endothelial CD202b marker expression; and
(l) endothelial CD31 marker expression.

As used herein, the term “probability” means a probability of 50% or more. Thus, a “probability” may mean a probability of 50% or more, 55% or more, 60% or more, 65% or more, 70% or more, 75% or more, 80% or more, 85% or more, 90% or more, or 95% or more.

As used herein, the term “treatment response” means a response by a subject receiving a treatment. The treatment response may be a positive response, where treatment has a positive effect on the cancer (e.g. shrinking tumor size or slowing growth) for which the subject is being treated; a negative response, where treatment has a negative effect on the cancer (e.g. increasing tumor size or speeding growth) for which the subject is being treated; or a neutral response, where treatment has no apparent effect on the cancer for which the subject is being treated. Alternatively, or in addition, a treatment response may be a change in progression free survival (PFS) or overall survival (OS), or both. The treatment response may be an increase in progression free survival (PFS) or overall survival (OS), or both. The treatment response may be a decrease in progression free survival (PFS) or overall survival (OS), or both.

In each of the methods of the invention, progression free survival (PFS) or overall survival (OS), or both, is over a period of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 months, or more. In one aspect of the invention, PFS or OS, or both, is over a period of at least about 24 months.

As used herein, the term “progression free survival (PFS)” is as defined by National Cancer Institute, i.e. the length of time during and after the treatment of a disease, such as cancer, that a patient lives with the disease but the disease does not get worse. PFS can also be defined as the length of time survived by a subject having cancer from a selected date, such as the date on which treatment began or the date on which pre-treatment blood is drawn to assess cancer progression, and where the cancer has not worsened or progressed.

As used herein, the term “overall survival (OS)” is as defined by National Cancer Institute, i.e. the length of time from either the date of diagnosis or the start of treatment for a disease, such as cancer, that patients diagnosed with the disease are still alive. OS is the length of time from when the treatment began or the date of the pre-treatment blood draw.

As used herein, the phrase “benefiting from the treatment” means that the subject receiving the treatment will experience an improvement in the condition being treated or a symptom of the condition being treated.

As used herein, the phrase “responding to the treatment” means the subject receiving the treatment will show an improvement the condition being treated or a symptom of the condition being treated.

As used herein, the term “pre-treatment sample” means a biological sample obtain from a subject before a particular treatment is administered to the subject. The particular treatment may not be the first or only treatment administered to the subject. However, it will be a treatment for which information regarding the treatment response in the subject is desired. As used herein, the term “post-treatment sample” means a biological sample obtain from the subject after a particular treatment is administered to the subject. If more than one treatment is administered to the subject, there may be more than one “post-treatment sample”.

As used herein, a “subject” is a human, a non-human primate, horse, cow, goat, sheep, a companion animal, such as a dog, cat or rodent, or other mammal.

As used herein, a “sample”, such as a patient sample, a pre-treatment sample, a post-treatment sample, is a biological sample and the sample may be, but is not limited to, one or more of peripheral blood, blood, lymph node, bone marrow, cerebral spinal fluid, tissue, urine, peripheral blood mononuclear cells (PBMCs), and cryopreserved PBMCs. When the biological sample is blood, the blood may be antecubital-vein blood, inferior-vena-cava blood, femoral vein blood, portal vein blood, or jugular-vein blood, for example. The sample may be a fresh sample or a properly prepared cryo-preserved sample that is thawed.

In each of the methods of the invention, it will be apparent that the amount of the sample in which the cells (CAMLs and CTCs) are assayed can vary. However, to obtain a relevant number of cells when the methods are based on determining the size of the cells, the sample should be at least about 2.5 mL. The amount of sample may also be at least about 3, 4, 5, 6, 7, 7.5, 8, 9, 10, 11, 12, 12.5, 13, 14, 15, 16, 17, 17.5, 18, 19, 20, 21, 22, 22.5, 23, 24, 25, 26, 27, 27.5, 28, 29, 30, 31, 32, 33, 34, 35, 36, 37, 38, 39, 40, 41, 42, 43, 44, 45, 46, 47, 48, 49, or 50 mL, or more. The amount of sample may also be between about 2.5 and 50 mL, about 2.5 and 20 mL, between about 5 and 15 mL, or between about 5 and 10 mL. In one aspect of the invention, the sample is about 7.5 mL. In certain aspects of the invention, the sample is a blood sample and the size of the blood sample is 7.5 mL.

With respect to methods for predicting OS and PFS of a subject having cancer based on circulating cell (CAML and CTC) numbers in a sample, a ratio of circulating cells in a selected volume of a sample from a subject having cancer is determined. It will be understood that various amounts of the sample can be used, but that a ratio of the number of cells in the sample to the size of the sample is determined and compared between subjects. For example, a ratio 8 cells in 15 mL of a sample is equivalent to 4 cells in 7.5 mL of a sample, and to 2 cells in 3.75 mL of a sample.

As used herein, “cancer” is a solid tumor, Stage I cancer, Stage II cancer, Stage III cancer, Stage IV cancer, carcinoma, sarcoma, neuroblastoma, melanoma, epithelial cell cancer, breast cancer, prostate cancer, lung cancer, pancreatic cancer, colorectal cancer, liver cancer, head and neck cancer, kidney cancer, ovarian cancer, esophageal cancer, uterine cancer, urothelial cancer, bladder cancer, endometrial cancer, cholangiocarcinoma, neuroendocrine cancer or other solid tumor cancer. The skilled artisan will appreciate that the methods of the invention are not limited to particular forms or types of cancer and that they may be practiced in association with a wide variety of cancers.

The subjects having cancer may be undergoing treatment. Reference to “treatment” or “treating” herein, in association with a subject having cancer, is a reference to any therapeutic molecule, substance, chemical, antibody, cell, device, agent, condition or procedure that can be used to either reduce growth or spread of the cancer, block growth or spread of the cancer, or cure the cancer. Suitable treatments include, but are not limited to, one or more of chemotherapy, single drug, combination of drugs, immunotherapy, targeted therapy, radiation therapy, chemoradiation, radiation combined with single or multiple drug, chemoradiation combined with single or multiple drugs, chemoradiation combined with single or multiple immunotherapeutics, cancer vaccine, and cell therapy. Suitable therapeutic molecules include, but are not limited to, atezolizumab, durvalumab and pembrolizumab. In a specific, non-limiting, example, the treatment is a cancer vaccine against breast cancer.

With respect to a change in the number or size of a CAML at the post-treatment from the pre-treatment, the difference may be a change in the size of the CAMLs, a change in the number of CAMLs, or both. The change in the size of the CAMLs may be a change in the average size calculated for all CAMLs in the samples, or the change in the size of the CAMLs may be a change in the size of the largest CAML in two or more the samples. The change in size may be an increase or a decrease. Because the shape of CAMLs can vary widely, it should be understood that the size of a CAMLs is calculated by measuring the distance between the two points on a cell that are the furthest apart. Where the CAMLs is round or approximately round, the size of the cell may be the diameter of the cell.

In each embodiment and aspect of the invention, cells may be isolated from the samples using size exclusion methodology, immunocapture, dendrimer-mediated multivalent cell capture, affinity based surface capture, biomimetic surface coating capture, selectin coated surfaces capture, other functionalized surface captures, inertial focusing chips, red blood cell lysis, white blood cell depletion, FICOLL separation, electrophoresis, dielectrophoresis, flow cytometry, magnetic levitation, and various microfluidic chips, or a combination thereof.

In one aspect of the invention, the CAMLs and CTCs may be isolated from the samples using size exclusion methodology that comprises using a microfilter. The pores have to be big enough to remove all red blood cells and majority of white blood cells. The pores have to be small enough to capture CAMLs and CTCs consistently. The microfilter may have a pore size ranging from about 5 microns to about 10 microns to capture both CTCs and CAMLs, and the microfilter may have a pore size ranging to about 20 microns to capture mainly CAMLs. In some instances, the pore sizes can be increased to about 50 μm, 60 μm, 70 μm, 80 μm, 90 μm, 100 μm or more, to capture only larger sized CAMLs. The pores of the microfilter may have a round, race-track shape, oval, square and/or rectangular pore shape. The microfilter may have precision pore geometry and/or uniform pore distribution.

In another aspect of the invention, the cells are isolated from the samples using a microfluidic chip via physical size-based sorting, hydrodynamic size-based sorting, grouping, trapping, immunocapture, concentrating large cells, or eliminating small cells based on size. The cell capture efficiency can vary depending on the collection method. The circulating cell (CAMLs and CTCs) size that can be captured on different platforms can also vary. The principle of using circulating cell size to determine prognosis and survival is the same, but the statistics will vary. Collection of circulating cells using CellSieve™ microfilters provides 100% capture efficiency and high quality cells.

In another aspect of the invention, the CAMLs and CTCs may be isolated from the samples using a microfluidic chip via physical size-based sorting, hydrodynamic size-based sorting, grouping, trapping, immunocapture, concentrating large cells, or eliminating small cells based on size.

In another aspect of the invention, blood is collected using a blood collection tube. CellSave blood collection tubes (Menarini Silicon Biosystems Inc.) provide stable cell morphology and size. Other available blood collection tubes do not provide cell stability. Cells can enlarge and may even burst collect in most other blood collection tubes.

In a further aspect of the invention, CAMLs and CTCs are isolated from the biological samples using a CellSieve™ low-pressure microfiltration assay.

In another aspect of the invention is to identify large cells in the sample without specifically identifying the cells as CAML cells per se, instead simply identifying the cells based on size of the cytoplasm and nucleus. Examples are techniques using color metric stains, such as H&E stains, or just looking at CK (+) cells.

### Example of Sample Collection and Processing

In each of the Examples provided herein and outlined below, peripheral blood was collected in CellSave Tubes (Menarini Silicon Biosystems Inc.) and processed within 96 hours. CellSieve™M microfiltration technique was used to collect all cancer associated cells (CTCs, EMTs, circulating endothelial cells (CECs), and CAMLs) in the blood sample. CellSieve™ microfilters have greater than 160,000 pores in uniform array with 7 μm pore diameter within a 9 mm area. The reagents include prefixation buffer, postfixation buffer, permeabilization buffer, and an antibody cocktail. The technique to perform the filtration used either a syringe pump set drawn at 5 mL/min or a vacuum pump [8]. The filtration process started by prefixing 7.5 mL of the blood in 7.5 mL of prefixation buffer before drawn through the filter. The filter and captured cells were then subjected to washing, postfixation, washing, permeabilization and washing. Then, the captured cells on the filter were stained with an antibody cocktail consisting of FITC-anti-Cytokeratin 8, 18, 19; Phycoerythrin (PE) conjugated EpCAM; and Cy5-anti-CD45 (5), followed by wash. Filters were placed onto a microscope slide and cover-slipped with Fluoromount-G/DAPI (Southern Biotech). An Olympus BX54WI Fluorescent microscope with Carl Zeiss AxioCam was used to image cells using specific fluorescent cubes and monochrome camera. Exposures were preset as 3 sec (Cy5), 2 sec (PE), 100-750 msec (FITC), and 10-50 msec (DAPI) for equal signal comparisons between cells. A Zen2011 Blue (Carl Zeiss) was used to process the images.

### EXPERIMENTAL EXAMPLES

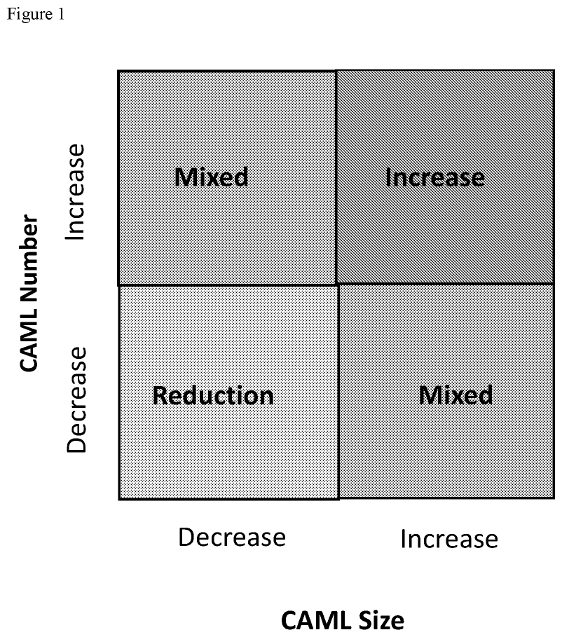

FIG. 1 explains the analysis of changes of CAML size and number between post-treatment and pre-treatment samples.

### Example 1

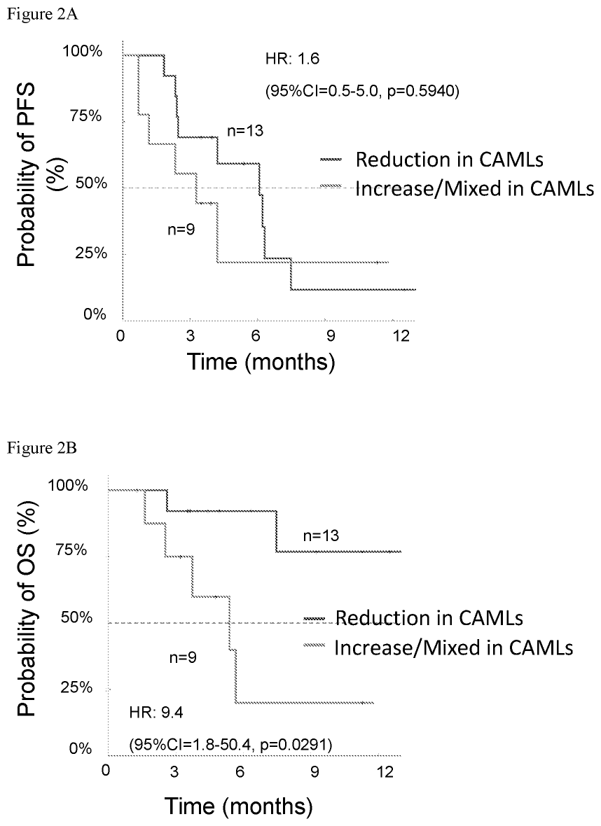

The following example illustrates a prediction of treatment response after treatment by humanized monoclonal antibody leronlimab of metastatic triple negative breast cancer (mTNBC). Leronlimab targets CCR5 markers in the tumor. An interim analysis of the drug response based on changes in CAML size and number was found to be predictive of the treatment response. In particular, a cohort of 22 patients provided baseline blood samples (pre-treatment) and blood samples after treatment. Of the 22 patients, 20 patients showed changes in the size of CAMLs and two patients did not show changes of CAML size, but had changes of CAML number. “Reduction in CAMLs” and “Increase/Mixed in CAMLs” in FIG. 2 are defined as shown in FIG. 1. Those patients with “Reduction in CAMLs” had increased PFS and OS in comparison to patients with “Increase/Mixed in CAMLs” (see FIG. 2A and 2B).

### Example 2

In a further experiment, an algorithm combining data from both CAMLs and CTCs can be applied to determine a patient's response to therapy, independent of the type of therapy.

The following is data from 83 metastatic breast cancer patients treated by six different therapies analyzed by the method described above. The treatments included:
Leronlimab as the base therapy, where some patients received Carbo, and others received Leronlimab, based on the prerogative of the attending oncologist
Bria Vax, based on the prerogative of the attending oncologist, then some received Pembro
Pembro with Binimetinib
Her2 therapy
Eribulin
Chemotherapy with trastuzumab and lapatinib

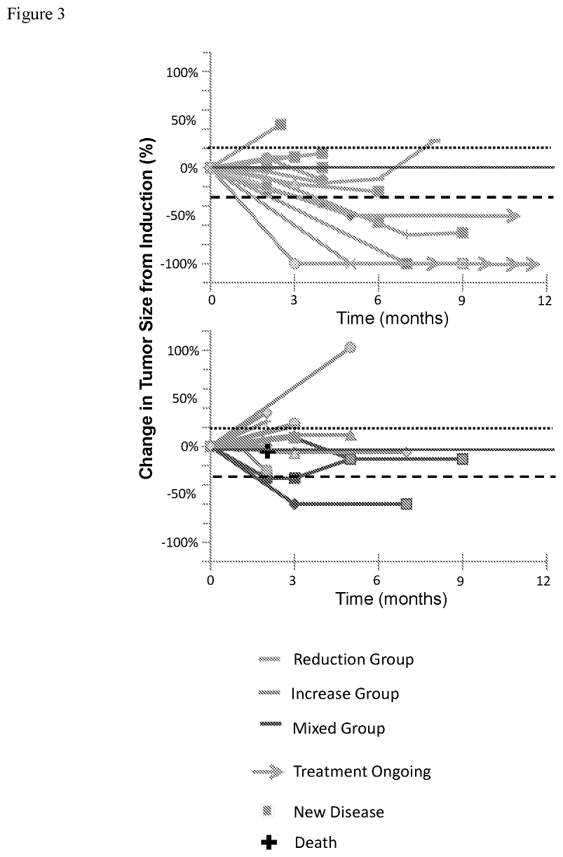

FIG. 3 shows a spider plot of change of tumor size, with known PET/CT scans (RECIST 1.1) with each group organized by color. As can be seen, there is a correlation between a reduction in tumor size and a reduction in CAML size and number.

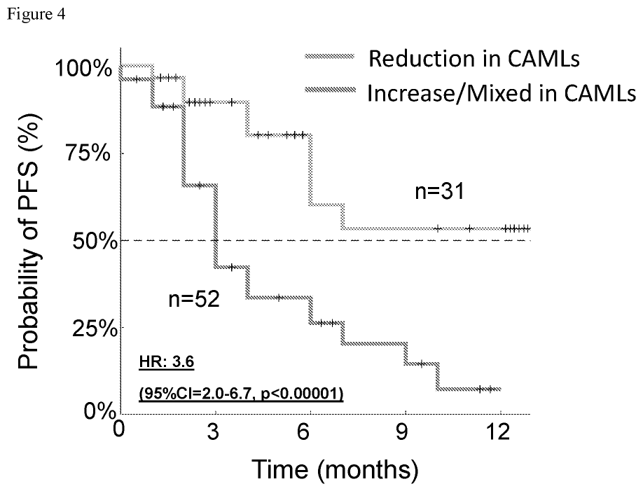

FIG. 4 is a Kaplan-Meier plot of PFS of the 83 metastatic breast cancer patients based on changes of CAMLs before and after induction of the therapies, with the data shown for “Reduction in CAMLs” and “Increase/Mixed in CAMLs” as described in FIG. 1. As can be seen, there is a correlation between PFS and a reduction in CAML size and number.

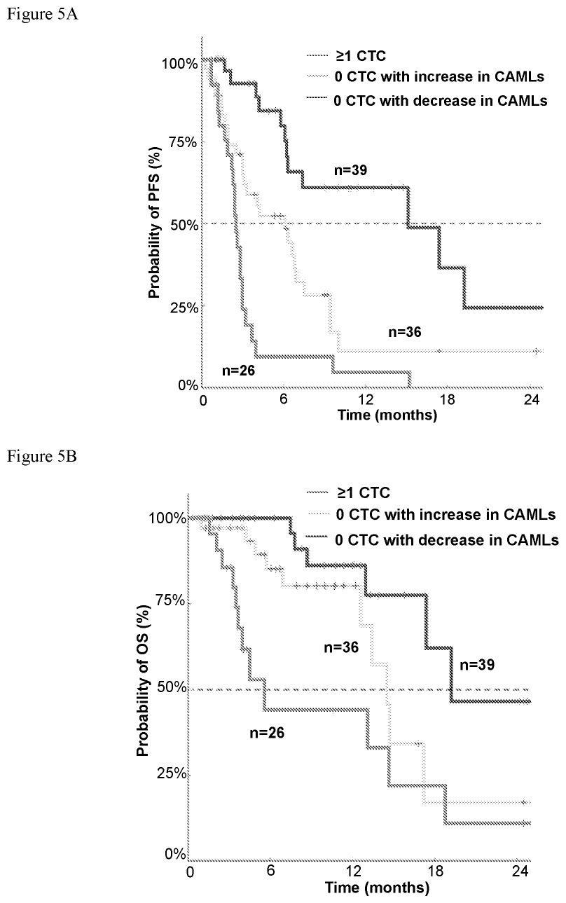

FIG. 5A is a Kaplan-Meier plot of PFS and FIG. 5B is a Kaplan-Meier plot of OS of the results from a prospective two year single blind multi-institutional study undertaken to evaluate CTCs and CAMLs changes before, and after, induction of a new line therapy in patients with metastatic breast cancer (mBC) (n=101). A baseline (BL) blood sample was taken prior to induction of a new therapy and a 2nd sample (T1) was taken after initiation of systemic therapy (˜30 days). LifeTracDx® liquid biopsy test was performed to collect CTCs and CAMLs using CellSieve™ microfilters. The quantities and subtypes of CTCs and CAMLs were analyzed. RECIST v1.1 was used to define progression free survival (PFS) for determining hazard ratios (HRs) by censored univariate and multivariate analysis at 2 years.

If the mBC patient had just one CTC in the sample (7.5 mL of blood) after induction of therapy, their PFS and OS were significantly shorter than those patients without any CTCs (FIGS. 5A and 5B). Patients with reduction of CAML size and number showed better PFS and OS than patients having a mixed result or an increase in CAML size and number (FIGS. 5A and 5B). In particular, CTCs were identified in 35% (n=35/101) of patients at baseline (BL) and 26% (n=26/101) at the post-treatment sample collection time point (T1). A single CTC at T1 was highly prognostic for worse PFS HR=6.5, p<0.0001 and OS HR=5.2, p=0.00188. CAMLs were found in 93% (n=94/11) of patients at BL and 86% (n=87/101) of patients at T1. CAML decreases (i.e. size/number) were significantly prognostic for improved PFS (HR=2.8, p=0.00029 and OS HR=2.6, p=0.01895 when CTCs were absent. Patients with ≥1 CTC at T1 (n=26) had median PFS=2.4 and mOS=4.7 months. Patients without CTCs plus increasing/mixed in CAMLs (n=36) had mPFS=5.9 and mOS=14.1 months. Patients without CTCs plus decrease in CAMLs (n=39) had mPFS=15.0 and mOS=18.8 months.

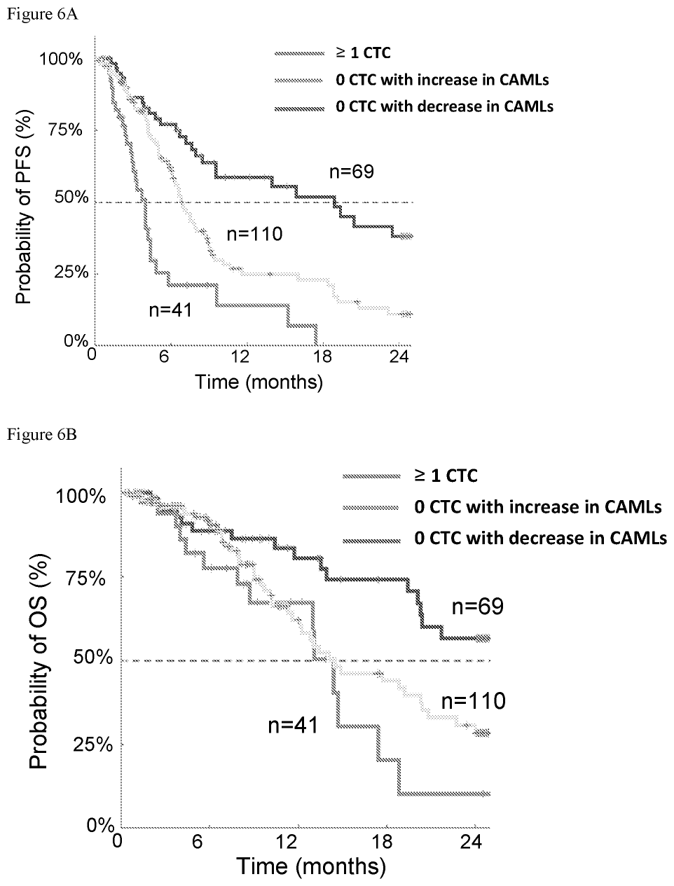

Furthermore, it has found that the algorithm used in FIG. 1 can be applied to a wide variety of solid tumors. FIG. 6A is a Kaplan-Meier plot of PFS and FIG. 6B is a Kaplan-Meier plot of OS of 220 metastatic cancer patients based on changes of CAMLs before and after induction of the therapies and presence of CTC at follow-up. The patients included those have the following cancers: breast, lung prostate, renal cell, pancreas, sarcoma and liver cancers. If they had a single CTC in the sample (7.5 mL of blood) after induction of therapy, their PFS and OS were significantly shorter than those patients without any CTCs. Patients with reduction of CAML size and number show better PFS and OS than patients having a mixed result or an increase in CAML size and number.

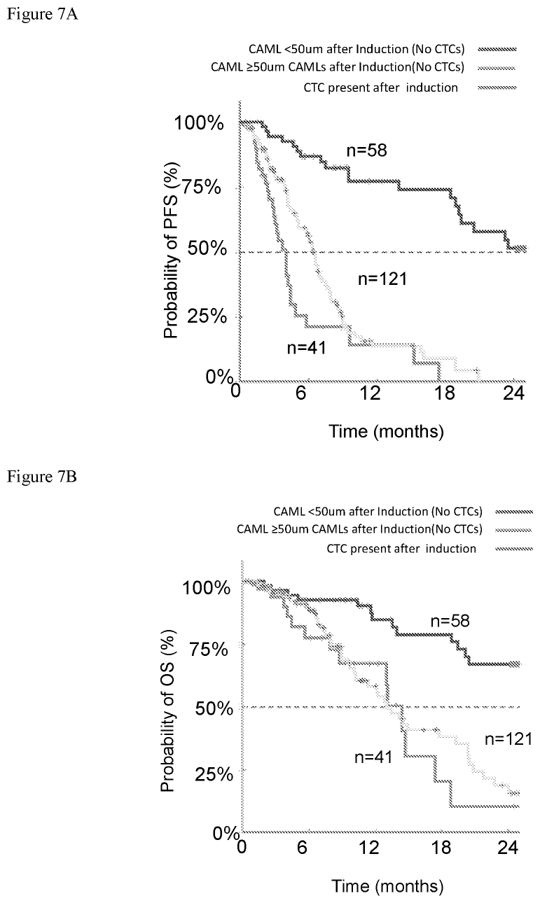

The same group of patient data used in FIG. 6 was reanalyzed based on CAML size ≥50 μm or <50 μm and presence of CTC at follow-up. FIG. 7A is a Kaplan-Meier plot of PFS and FIG. 7B is a Kaplan-Meier plot of OS of the same 220 metastatic cancer patients as FIG. 6. After induction of therapy, patients having either CAMLs >50 μm or at least one CTC in the sample have poor PFS and OS.

### Example 3

A TNBC (triple negative breast cancer) clinical trial with the drug targeting CCR5 marker on the tumor was conducted. CellSieve™ assay was performed, providing information about CTCs and CAMLs including staining for the CCR5 marker on these cells. The CCR5 drug does not kill the tumor. At the end of the clinical trial, initial analysis of the data showed combinations of different sets of information can provide useful stratification of drug response.

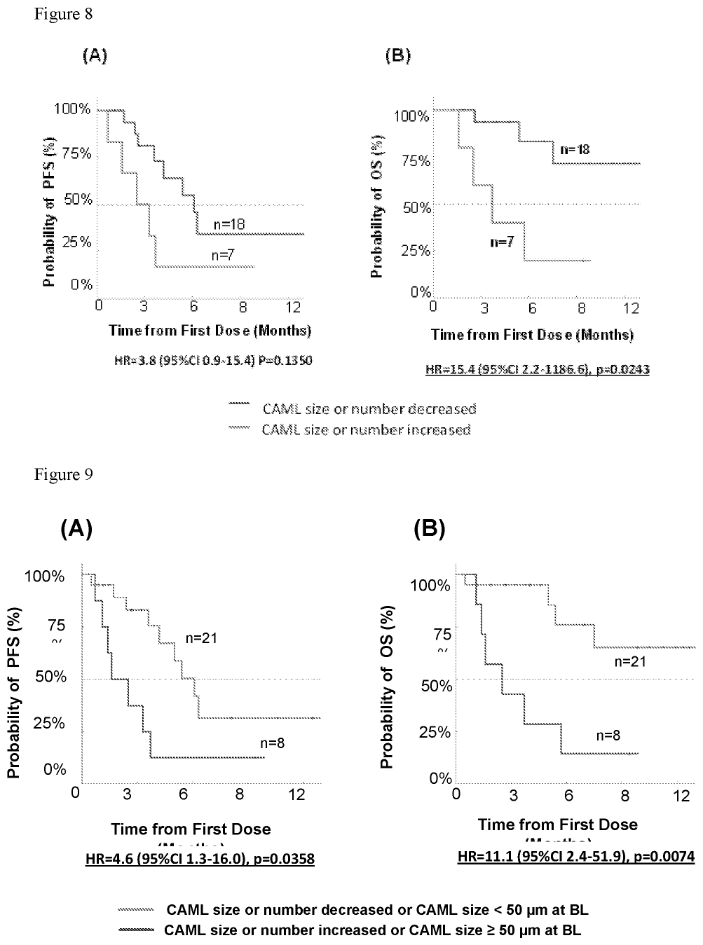

Decrease of CAML size and decrease of CAML number ˜30 days after first treatment cycle of a CCR5 drug compared to the baseline provided informative stratification of responders and non-responders to the drug as shown in FIG. 8A for PFS and 8B for OS of 25 patients.

Four patients did not have a 30 day follow-up blood sample after first treatment of CCR5 drug. Since patients with CAML <50 μm tend to have high probability of doing better than patients with larger CAMLs, the CAML size at the baseline of these four patients were include in the following way. Patients without 30 day follow-up and had all CAML <50 μm were grouped with decrease of CAML size or decrease of CAML number of the 18 patients in 30 days after first treatment cycle of CCR5 drug. The revised PFS and OS are plotted in FIGS. 9A and 9B, respectively.

These two examples demonstrate CellSieve™M blood test can provide more useful information about the cancer and/or the response to drug than just analyzing a single marker alone.

### Example 4

CAMLs are prevalent in the circulation of non-small cell lung carcinoma (NSCLC) patients, appearing as giant phagocytic macrophages that represent an inflammatory pro-tumorigenic microenvironment. Previously it was shown that patients with engorged CAMLs of ≥50 μm after treatment are prognostic for poor clinical outcomes. However, analyzing the dynamic changes in CAMLs over time or in response to treatment, i.e. chemoradiation (CRT) and immunotherapy (IMT) has not been evaluated. Monitoring of n=182 unresectable NSCLC stage II/III patients treated with CRT alone (n=91) or with concurrent IMT (n=91) was performed to evaluate changes in CAMLs before and after CRT induction at it relates to progression free survival (PFS) or overall survival (OS).

In particular, patients were prospectively procured from 3 different regimes, treated with CRT alone (n=91), treated concurrently with CRT & Atezolizumab (n=40, clinical trial NCT02525757), or treated concurrently with Durvalumab (n=51). 182 patients with pathologically confirmed stage II/III unresectable NSCLC were recruited. A total of 15 mL blood samples were drawn prior to start of therapy at baseline (BL) and ˜5 weeks (T1) after CRT induction. Blood filtration was done using CellSieve™ filters, then CAMLs were identified and measured to evaluate PFS & OS hazard ratios (HRs) by censored univariate and multivariate analyses at 2 years. For analysis, patients were compared based on CAML size increases greater than thresholds versus patients with any changes below the threshold, including decreases and no differences in CAML change.

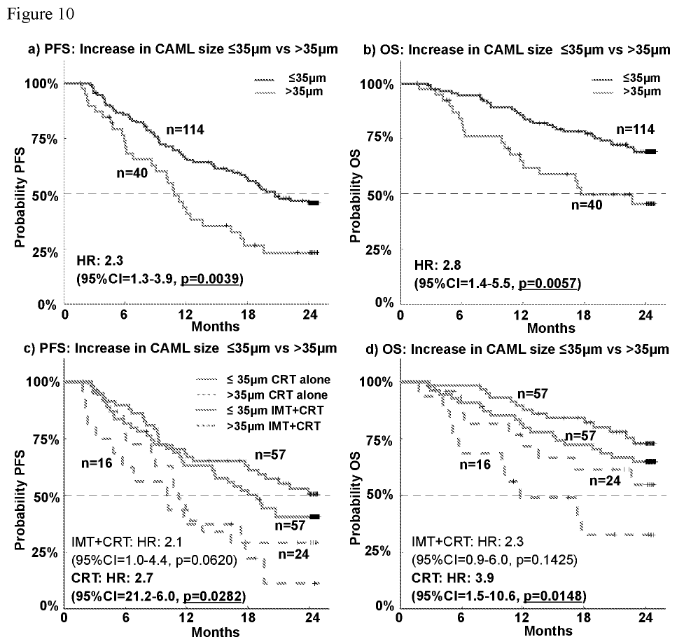

The results demonstrated that increases in CAML size of 10-50 μm between BL and T1 time points correlated with increasingly shorter PFS and OS (data not shown). Increases in CAML size of 35 micron between BL and T1 was optimal in stratifying patients in terms of PFS and OS (FIG. 10A and 10B). Increases in CAML size of 35 micron significantly stratified patients by PFS and OS treated with CRT but not significantly for patients treated with IMT ((FIG. 10C and 10D).

### Example 5

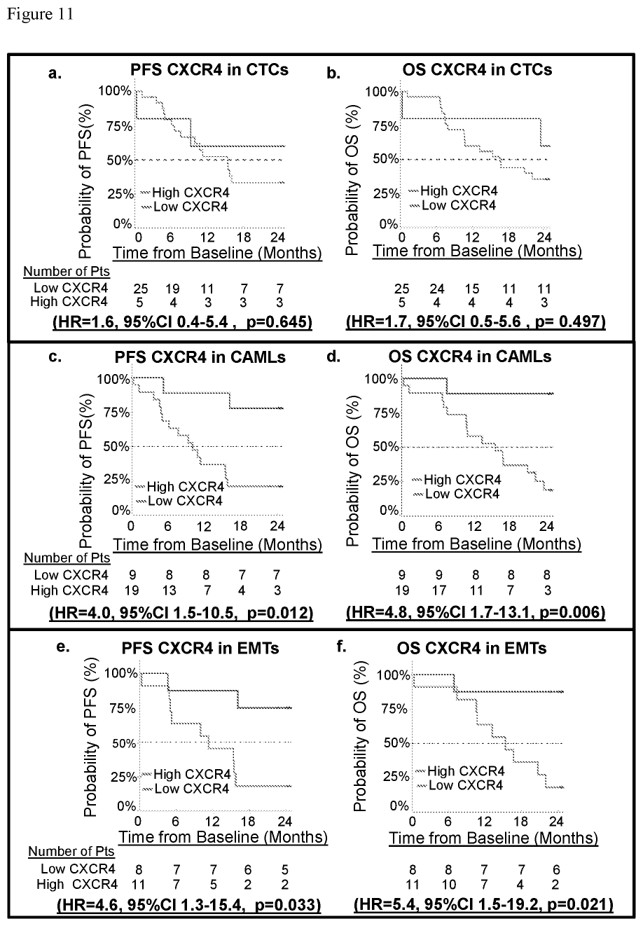

In addition to CAML size and number, some of the tumor properties can also be indicative of the aggressiveness of the cancer. An example is the marker CXCR4. Peripheral blood from 30 pancreatic cancer patients was analyzed for CXCR4 expression. Kaplan-Meier plots of PFS and OS for CTCs, CAMLs, and EMT were analyze for CXCR4 expression. CXCR4 in CTCs was not found to be a significant predictor of progression (HR=1.6, 95% CI 0.4-5.4, p=0.645) (FIG. 11A) nor OS (HR=1.7, 95% CI 0.5-5.6, p=0.497) (FIG. 11B). However, higher CXCR4 expression in CAMLs was related to significantly faster progression (HR=4.0, 95% CI 1.5-10.5, p=0.012) (FIG. 11C) and significantly higher mortality (HR=4.8, 95% CI 1.7-13.1, p=0.006) (FIG. 11D). In addition, expression of CXCR4 on EMTs also appear to relate to progression and survival, with patients with high CXCR4 expressing EMTs having significantly faster progression (HR=4.6, 95% CI 1.3-15.4, p=0.033) (FIG. 11E) and higher mortality rate (HR=5.4, 95% CI 1.5-19.2, p=0.021) (FIG. 11F).

Thus, CXCR4 expression can be used to further stratify the patients in the CAML <50 μm group or the CAML reduction group who are going to progress quickly or die sooner.

### Example 6

A single blind, multi-year prospective study was undertaken to test the relationship of PD-L1 expression and CAMLs size to obtain PFS/OS in non-small cell lung cancer (NSCLC). 96 patients participated in chemoradiotherapy (CRT) followed by immunotherapy. This included atezolizumab (prospective single arm NCT02525757) n=39, durvalumab n=52 and pembrolizumab n=5. Blood samples (7.5 mL) were taken at baseline (BL), at CRT completion (T1), and at ˜1 month after CRT (T2) after immunotherapy. Blood was filtered by CellSieve™ filtration. CAMLs were analyzed for their size and PD-L1 expression. The PFS and OS data were analyzed after 1-2 treatment cycles of immunotherapy after CRT, and the number of patients at T2 time point dropped to 80.

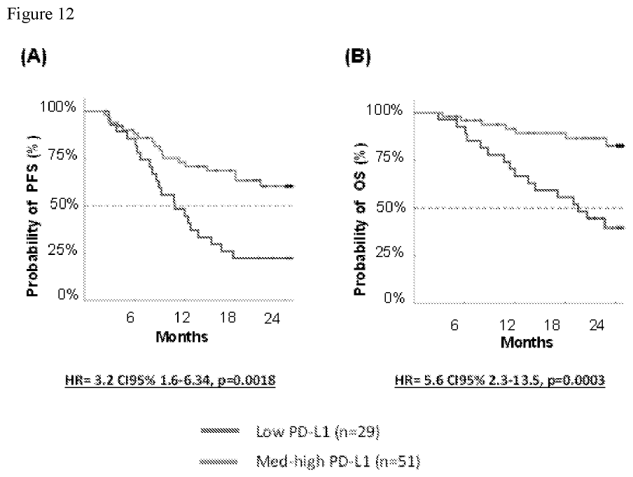

The patients with just one large CAML ≥50 μm in 7.5 mL of blood had shorter PFS and OS (data not shown). Because this study was on immunotherapy, the PD-L1 expression provided better stratification of PFS and OS as shown in FIG. 12. The patients with medium to high PD-Ll expression benefited significantly from the immunotherapy.

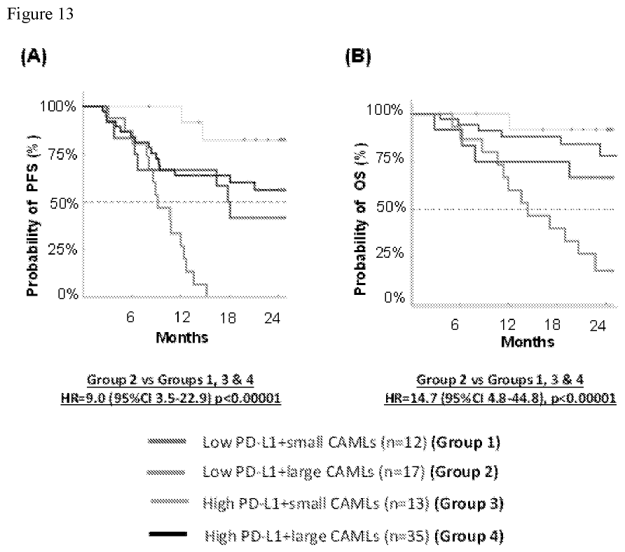

Since both CAML size and PD-L1 expression were obtained from the same CellSieve™ assay, the data could also be analyzed together into four categories: (1) low PD-L1 and small CAMLs, (2) low PD-L1 and large CAMLs, (3) high PD-L1 and small CAMLs, and (4) high PD-L1 and large CAMLs. The term “large CAMLs” means, one or more CAML in the sample is >50 μm. The term “small CAMLs” means all CAMLs in the sample are <50 μm. The patients with low PD-L1 and large CAMLs had significantly worse PFS and OS as shown in FIG. 13. For PFS, the HR=9.0 (95% Cl 3.5-22.9) and p<0.00001 comparing group 2 versus groups 1, 3 and 4 together. For OS, the HR=14.7 (95% Cl 4.8-44.81) and p<0.00001 comparing group 2 versus groups 1, 3 and 4 together.

This assay was very useful to determine the non-responders to immunotherapy just after 1-2 immunotherapy treatments.

### Example 7

CAMLs appear to parallel the real time inflammatory PD-L1 state of the tumor microenvironment. Previously, it was demonstrated in local non-small cell lung carcinoma (NSCLC) that the PD-L1 expression on CAMLs is dynamic and can predict response to PD-L1/PD-1 immunotherapies (IMTs) following sequential sampling, and after chemotherapy induction (˜30 days) based on progression free survival (PFS) & overall survival (OS). However, this had not been tested in recurrent NSCLC. Therefore, PD-L1 expression was monitored in CAMLs before and after chemotherapy induction (˜30 days) to evaluate CAMLs' PD-L1 predictive value in recurrent NSCLC pts treated with or without IMT.

A single blind multi-year prospective study was undertaken to test the relationship of PD-L1 expression in CAMLs to PFS and OS, pre and post chemotherapy induction in recurrent mNSCLC, with (n=41) or without (n=41) additional anti-PD-L1/PD-1 IMT. This included three IMTs: atezolizumab (n=4), nivolumab (n-8) and pembrolizumab (n=29). 82 patients with pathologically confirmed recurrent NSCLC prior to treatment for newly recurrent disease were recruited. Blood samples (15 mL) were taken at Baseline (BL), prior to chemotherapy, and ˜30 days after chemotherapy (T1). Blood was filtered by CellSieve™ filtration and CAMLs expression was broken into a binary high or low score, to evaluate PFS and OS hazard ratios (HRs) by censored univariate and multivariate analysis at 24 months.

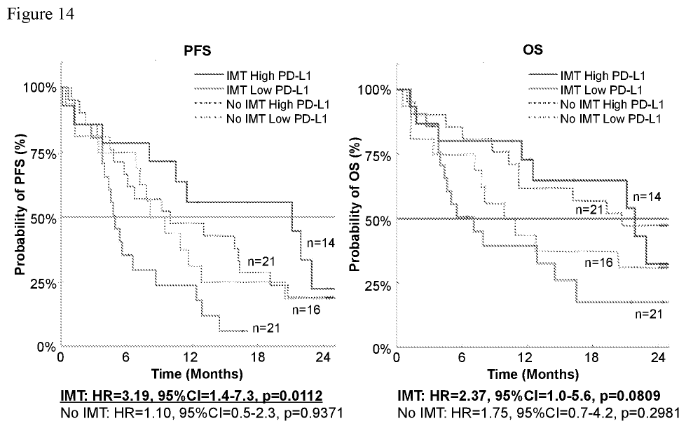

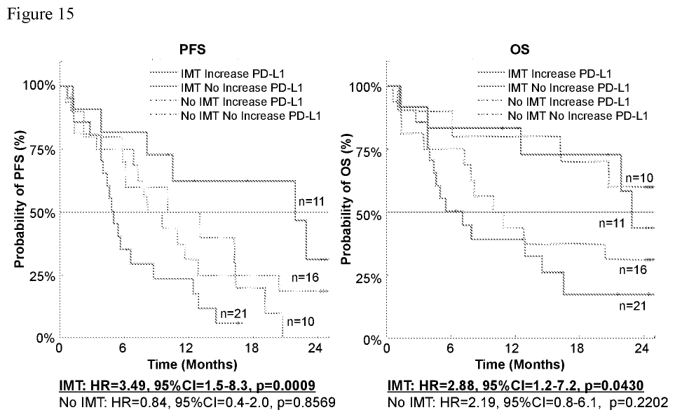

The results (FIGS. 14 and 15) show CAMLs were found in 97% of all available samples, 94% at BL and 100% at T1. At BL, high PD-L1 in patients not treated with IMT was not significant for PFS (p-0.825) nor OS (p-0.518). At T1, patients with high PD-L1 in patients not treated with IMT was not significant for PFS (HR=1.10, p=0.937) nor OS (HR=1.75 p=0.298). At T1, patients with high PD-L1 treated with IMT had significantly better PFS (HR=3.19, p=0.0112), and borderline OS (HR=2.37, p=0.0809). Patients with increased PD-L1 expression between BL & T1 had significantly better PFS (HR=3.49, p=0.0009) and OS (HR=2.88, p=0.0430).

Thus, in recurrent NSCLC, high PD-L1 expression in CAMLs is prognostic with IMT use and predicts for response to consolidated IMT after CRT. Monitoring dynamic changes of PD-L1 in CAMLs appears to predict immunotherapy effectiveness in recurrent NSCLC

### CITATIONS

1. Adams, D., et al., Circulating giant macrophages as a potential biomarker of solid tumors. PNAS 2014, 111 (9): 3514-3519.
2. International Patent Appln. Publn. No. WO 2013/181532, dated Dec. 5, 2013.
3. International Patent Appln. Publn. No. WO 2016/33103, dated Mar. 3, 2016.
4. International Patent Appln. Publn. No. WO 2018/151865, dated Aug. 23, 2018.
5. International Patent Appln. Publn. No. WO 2019/178226, dated Sep. 19, 2019.
6. International Patent Appln. Publn. No. WO 2018/184005, dated Oct. 4, 2018.
7. Ballman, K. V., Biomarker: Predictive or Prognostic? J. Clin. Oncol. 2015, 33 (33): 3968-3972.
8. Adams D L, et al. The systematic study of circulating tumor cell isolation using lithographic microfilters. RSC Adv 2014, 4:4334-4342.

1. A method for predicting a treatment response in a subject having cancer, comprising determining and comparing the size and number of CAMLs in samples obtained from the subject before treatment (pre-treatment sample) and after treatment (post-treatment sample), and making a prediction based thereon,
wherein when at least one CAML in the post-treatment sample is greater in size than the largest CAML in the pre-treatment sample and wherein there are more CAMLs in the post-treatment sample than in the pre-treatment sample, the subject is predicted to not respond to treatment.

2. A method for predicting a treatment response in a subject having cancer, comprising obtaining at least one pre-treatment sample from a subject having cancer and obtaining at least one post-treatment sample from the subject, and comparing the number and size of CAMLs between the pre-and post-treatment samples for differences,
wherein when the largest CAML in the post-treatment sample is decreased in size or similar in size in comparison to the largest CAML in the pre-treatment sample, and the number of CAMLs in the post-treatment sample is less than the number of CAMLs in the pre-treatment sample, the subject has a probability of benefiting from the treatment, or responding to the treatment.

3. A method for predicting a treatment response in a subject having cancer, comprising obtaining at least one pre-treatment sample from a subject having cancer and obtaining at least one post-treatment sample from the subject, and comparing the number and size of CAMLs between the pre-and post-treatment samples for differences,
wherein when the largest CAML in the post-treatment sample is decreased in size or similar in size in comparison to the largest CAML in the pre-treatment sample, and the number of CAMLs in the post-treatment sample is more than or the same as the number of CAMLs in the pre-treatment sample, the subject has a probability of not benefiting from the treatment, or not responding to the treatment.

4. A method for predicting a treatment response in a subject having cancer comprising obtaining at least one pre-treatment sample from a subject having cancer and obtaining at least one post-treatment sample from the subject, and comparing the number and size of CAMLs between the pre-and post-treatment samples for differences,
wherein when the largest CAML in the post-treatment sample is increased in size or similar in size in comparison to the largest CAML in the pre-treatment sample, and the number of CAMLs in the post-treatment sample is less than or the same as the number of CAMLs in the pre-treatment sample, the subject has a probability of not benefiting from the treatment, or not responding to the treatment.

5. A method for predicting a treatment response in a subject having cancer comprising obtaining at least one pre-treatment sample from a subject having cancer and obtaining at least one post-treatment sample from the subject, and comparing the number and size of CAMLs between the pre-and post-treatment samples for differences,
wherein when the largest CAML in the post-treatment sample is increased in size or similar in size in comparison to the largest CAML in the pre-treatment sample, and the number of CAMLs in the post-treatment sample is more than the number of CAMLs in the pre-treatment sample, the subject has a probability of not benefiting from the treatment, or not responding to the treatment.

6. A method for predicting a treatment response in a subject having cancer, comprising obtaining at least one post-treatment sample from a subject having cancer, and assaying the sample for CTCs, wherein when one or more CTCs are detected in the sample, the subject has a probability of not benefiting from the treatment, or not responding to the treatment.

7. A method for predicting a treatment response in a subject having cancer, comprising obtaining at least one pre-treatment sample from a subject having cancer and obtaining at least one post-treatment sample from the subject, and (i) comparing the number and size of CAMLs between the pre-and post-treatment samples for differences and (ii) determining the number of CTCs in the post-treatment samples, wherein when a difference in CAML number and/or size is found and/or at least one CTC is detected, a treatment response is predicted.

8. The method of claim 7, wherein when at least one CTC is detected in the post-treatment sample, the subject has a probability of not benefiting from the treatment, or not responding to the treatment.

9. The method of claim 1, wherein the CAMLs have the following characteristics:
(a) multiple individual nuclei and/or one or more fused nuclei having a size of about 14-64μ m;
(b) cell size of about 20-300 μm in size; and
(c) morphological shape selected from the group consisting of spindle, tadpole, round, oblong, two legs, more than two legs, thin legs, and amorphous.

10. The method of claim 9, wherein the CAMLs have one or more of the following additional characteristics:
(d) CD14 positive phenotype;
(e) CD45 expression;
(f) EpCAM expression;
(g) vimentin expression;
(h) PD-L1 expression;
(i) monocytic CD11C marker expression;
(j) endothelial CD146 marker expression;
(k) endothelial CD202b marker expression;
(l) endothelial CD31 marker expression; and
(m) epithelial cancer cell CK8, 18, and/or 19 marker expression.

11. The method of claim 1, wherein the size of the sample is between 5 and 50 mL.

12. The method of claim 1, wherein the source of the sample is one or more of peripheral blood, blood, lymph node, bone marrow, cerebral spinal fluid, tissue, urine, peripheral blood mononuclear cells (PBMCs), and cryopreserved PBMCs.

13. The method of claim 12, wherein the sample is antecubital-vein blood, inferior-vena-cava blood, femoral vein blood, portal vein blood, or jugular-vein blood.

14. The method of claim 1, wherein the cancer is a solid tumor, Stage I cancer, Stage II cancer, Stage III cancer, Stage IV cancer, carcinoma, sarcoma, neuroblastoma, melanoma, epithelial cell cancer, breast cancer, prostate cancer, lung cancer, pancreatic cancer, colorectal cancer, liver cancer, head and neck cancer, kidney cancer, ovarian cancer, esophageal cancer, uterine cancer, urothelial cancer, bladder cancer, endometrial cancer, cholangiocarcinoma, neuroendocrine cancer or other solid tumor cancer.

15. The method of claim 1, wherein CAMLs and CTCs are isolated from the sample using one or more means selected from the group consisting of size exclusion methodology, immunocapture, dendrimer-mediated multivalent cell capture, affinity based surface capture, biomimetic surface coating capture, selectin coated surfaces capture, other functionalized surface captures, inertial focusing chips, red blood cell lysis, white blood cell depletion, FICOLL separation, electrophoresis, dielectrophoresis, flow cytometry, magnetic levitation, and various microfluidic chips, or a combination thereof.

16. The method of claim 15, wherein CAMLs and CTCs are isolated from the samples using size exclusion methodology that comprises using a microfilter.

17. The method of claim 16, wherein the microfilter has a pore size ranging from about 5 microns to about 20 microns.

18. The method of claim 17, wherein the pores of the microfilter have a round, race-track shape, oval, square and/or rectangular pore shape.

19. The method of claim 17, wherein the microfilter has precision pore geometry and uniform pore distribution.

20. The method of claim 15, wherein CAMLs and CTCs are isolated using a microfluidic chip via physical size-based sorting, hydrodynamic size-based sorting, grouping, trapping, immunocapture, concentrating large cells, or eliminating small cells based on size.

21. The method of claim 1, wherein CAMLs and CTCs are isolated from the samples for the determining steps using a low-pressure microfiltration assay.

22. The method of claim 1, wherein the treatment is one or more of chemotherapy, single drug, combination of drugs, immunotherapy, targeted therapy, radiation therapy, chemoradiation, radiation combined with single or multiple drug, chemoradiation combined with single or multiple drugs, cancer vaccine, and cell therapy.

23. The method of claim 22, wherein the treatment is a cancer vaccine.
